(19) United States
(12) Patent Application Publication
(10) Pub. No.: US 2024/0408363 A1
(43) Pub. Date: Dec. 12, 2024

(54) NITRIC OXIDE FORMULATION DELIVERY DEVICE AND METHOD OF USE

(71) Applicant: NOxy Health Products, Inc., Anaconda, MT (US)

(72) Inventors: C. Michael MILLER, Anaconda, MT (US); David A. BELL, Farmington, UT (US)

(21) Appl. No.: 18/736,145

(22) Filed: Jun. 6, 2024

Related U.S. Application Data
(60) Provisional application No. 63/471,327, filed on Jun. 6, 2023.

(51) Int. Cl. A61M 35/00; A61K 9/00; A61K 9/12; A61K 31/04

(52) U.S. Cl. CPC A61M 35/006; A61K 9/0014; A61K 9/122; A61K 31/04

(57) ABSTRACT

A nitric oxide (NO) formulation delivery device comprising a first cartridge containing a first liquid solution and a first foaming device configured to transform the first liquid solution into a first foam solution, a second cartridge containing a second liquid solution and a second foaming device configured to transform the second liquid solution into a second foam solution, a plenum in fluid communication with the first cartridge and the second cartridge, the plenum being configured to combine the first foam solution and the second foam solution to create a combined turbulent foam solution, and a nozzle in fluid communication with the confluence portion and configured to uniformly discharge the combined turbulent foam solution.

### CROSS-REFERENCE TO RELATED APPLICATIONS

This application claims priority to U.S. Provisional Application Ser. No. 63/471,327, filed Jun. 6, 2023, the entire contents of which are incorporated by reference herein.

### FIELD

The present disclosure relates generally to exogenous nitric oxide formulation and delivery and its devices and methods of use.

### BACKGROUND

This section provides background information related to the present disclosure which is not necessarily prior art.

Nitric Oxide (NO) is a molecular compound or enzyme, that is naturally occurring in vascular systems of the body, and when available in sufficient concentration plays a key role in the dynamic “Molecular and Cellular Skin Wound Healing Processes.”

In its initial formulation in the epithelial layers of all blood vessels, this endogenous enzyme, or Endothelial Nitric Oxide Synthase, “eNOS,” formulates as a “signaling molecule” where it is dispensed by vascular flows, and can motivate important cellular functions found in the “Vascular Response Cascade” associated with wound healing, including vasodilation, contraction and clotting, closure, and nitric oxide enzyme-guided, protective inflammation. When appropriately activated and sustained by a sufficient concentration of enzymatic nitric oxide, it is a powerful promotor of improved oxygenation and nutrient delivery, as well as an anti-pathogenic within the biosphere of wounded tissues.

Likewise, when stimulated by excessive inflammation, NO is actively transformed to an inductive form of the NO synthase or “iNOS,” where it can terminate inflammatory neutrophilic and macrophagic functions and actively target and dismantle pathogens associated with chronic wound development leading up to scar formation, renewed cellular growth, and epithelial healing requisite to wound contraction and wound scar remodeling.

But sometimes due to age, and/or concomitant disease, the body lacks sufficient NO concentration to produce and deliver sufficient synthase-driven nitric oxide on its own in order to overcome the initial stages of increasing inflammation which lead up to chronic wounding. In such cases, an alternative exogenous NO supplementation may be required.

Even so, NO supplementation poses significant challenges primarily associated with its short half-life (often quoted as less than 3 seconds), and its proclivity to instantaneous react with ambient oxygen to form Nitrogen Dioxide (NO₂), which in turn can further complicate the storage, transportation, and wound site application of NO. Moreover, methods for motivating the diffusion of NO directly into the wound poses significant challenges.

### SUMMARY

This section provides a general summary of the means and methods associated with the invention and is not a comprehensive disclosure of its full scope or of all its features.

One aspect of the disclosure provides a nitric oxide (NO) formulation delivery device comprising a first cartridge containing a first liquid solution and a first foaming device configured to transform the first liquid solution into a first foam solution, a second cartridge containing a second liquid solution and a second foaming device configured to transform the second liquid solution into a second foam solution, a plenum comprising (i) a first leg portion in fluid communication with the first cartridge, (ii) a second leg portion in fluid communication with the second cartridge, and (iii) a confluence portion in fluid communication with the first leg portion and the second leg portion and configured to combine the first foam solution and the second foam solution to create a turbulent combined foam solution, and a nozzle in fluid communication with the confluence portion and configured to discharge the combined foam solution.

Implementations of the disclosure may include one or more of the following optional features. In some implementations, the first leg portion is configured to receive the first foam solution, and the first leg portion includes one or more flow tripper obstructions configured to disrupt the flow of the first foam solution. The second leg portion may be configured to receive the second foam solution, and the second leg portion may include one or more flow tripper obstructions configured to disrupt the flow of the second foam solution.

The first solution may comprise at least one of water, a nitrite such as sodium nitrite, and a cationic surfactant. The second solution may comprise at least one of water, a cationic surfactant, and a suitable acidic component such as citric acid.

The plenum and nozzle may be configured to discharge a consistent dose of the combined foam solution. The consistent dose of the combined foam solution may be between approximately 0.6 g and 1.0 g.

The NO formulation delivery device may further comprise a mixer disposed between the confluence portion of the plenum and the nozzle, the mixer comprising an agitator designed to yield a combined foam solution into a turbulent flow domain characterized by a Reynolds number of at least 2100 as it passes through at least one complete revolution through the mixer section. The mixer may also include a flow-straightening portion disposed between the agitator and the nozzle, the flow-straightening portion configured to better provide a uniformly turbulent, combined foam solution. The mixer may also be designed to prevent the combined foam solution from reversing its flow back into the plenum.

The NO formulation delivery device may further comprise a base configured to receive the first cartridge and the second cartridge. The base may include a catch basin disposed below the nozzle, the catch basin configured to receive any residual combined foam or liquid solution discharged from the nozzle.

The plenum may include a head portion connecting the first leg portion to the second leg portion.

One aspect of the disclosure provides a nitric oxide (NO) formulation delivery device comprising a first cartridge containing a first liquid solution and a first foaming device configured to transform the first liquid solution into a first foam solution, a second cartridge containing a second liquid solution and a second foaming device configured to transform the second liquid solution into a second foam solution, a plenum in fluid communication with the first cartridge and the second cartridge, the plenum being configured to combine the first foam solution and the second foam solution to create a combined turbulent foam solution, one or more liquid pumps configured to operate automatically in response to motion detected by one or more sensors signaling the first and second pumps to pump the first liquid solution and the second liquid solution from the first cartridge and the second cartridge to the plenum, and a nozzle in fluid communication with the confluence portion and configured to discharge the combined foam solution therefrom.

Implementations of the disclosure may include one or more of the following optional features. In some implementations, the first solution comprises water, a nitrite such as sodium nitrite, and a cationic surfactant. The second solution may comprise at least one of water, a cationic surfactant, and a reactive acidic component such as citric acid.

The plenum and nozzle may be configured to discharge a consistent dose of the combined foam solution. The consistent dose of the combined foam solution may be between approximately 0.6 g and 1.0 g.

One aspect of the disclosure provides a method comprising providing a nitric oxide (NO) formulation delivery device comprising (i) a first cartridge containing a first liquid solution and a first foaming device configured to transform the first liquid solution into a first foam solution, (ii) a second cartridge containing a second liquid solution and a second foaming device configured to transform the second liquid solution into a second foam solution, (iii) a plenum in fluid communication with the first cartridge and the second cartridge, the plenum being configured to combine the first foam solution and the second foam solution to create a combined foam solution, and (iv) a nozzle in fluid communication with the confluence portion and configured to discharge the combined foam solution, simultaneously converting the first liquid solution from the first cartridge into the first foam solution, converting the second liquid solution from the second cartridge into the second foam solution, and pumping the first foam solution and the second foam solution into the plenum, mixing the first foam solution and the second foam solution in the plenum to create the combined foam solution, and discharging the combined foam solution via the nozzle.

Implementations of the disclosure may include one or more of the following optional features. In some implementations, the method further comprises topically applying the combined foam solution to a skin wound.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected configurations and not all possible implementations and are not intended to limit the scope of the present disclosure.

FIG. 1A is a perspective view of an exemplary nitric oxide formulation delivery device in accordance with the principles of the present disclosure;

FIG. 1B is a front view of the nitric oxide formulation delivery device of FIG. 1A;

FIG. 1C is a side view of the nitric oxide formulation delivery device of FIG. 1A;

FIG. 1D is a cross-sectional view of nitric oxide formulation delivery device of FIG. 1A, taken along line 1D-1D in FIG. 1C;

FIG. 2A is a perspective view of a plenum of the nitric oxide formulation delivery device of FIG. 1A;

FIG. 2B is a front view of the plenum of FIG. 2A;

FIG. 3A is a side view of a mixer of the nitric oxide formulation delivery device of FIG. 1A;

FIG. 3B is a cross-sectional view of the mixer of FIG. 3A, taken along line 4B-4B in FIG. 4A;

FIG. 3C is a top view of the mixer of FIG. 3A;

FIG. 4 is a perspective view of a stand of the nitric oxide formulation delivery device of FIG. 1A;

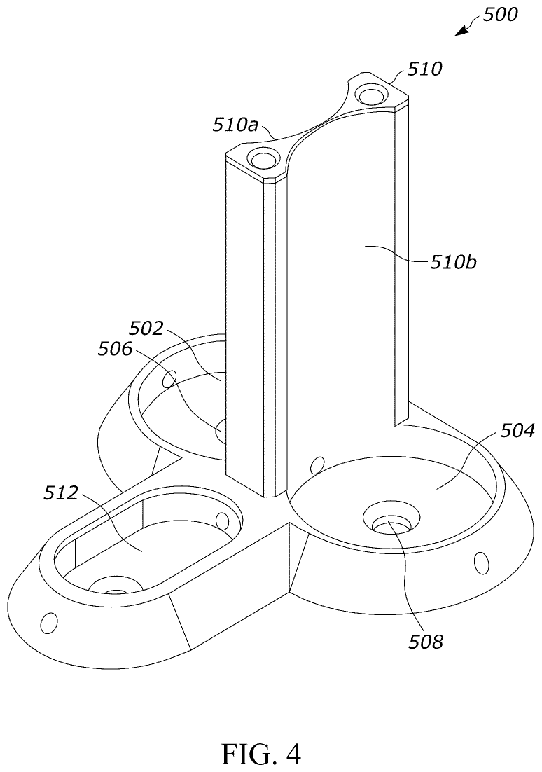

FIG. 5 is a schematic view of an automatic nitric oxide formulation delivery device using a cam pump in accordance with the principles of the present disclosure;

FIG. 6 is a schematic view of an automatic nitric oxide formulation delivery device using a liquid pump in accordance with the principles of the present disclosure; and

FIG. 7 is an exemplary method of using the nitric oxide formulation delivery device of FIG. 1A in accordance with principles of the present disclosure.

Corresponding reference numerals indicate corresponding parts throughout the drawings.

### DETAILED DESCRIPTION

Example configurations will now be described more fully with reference to the accompanying drawings. Example configurations are provided so that this disclosure will be thorough and will fully convey the scope of the disclosure to those of ordinary skill in the art. Specific details are set forth such as examples of specific components, devices, and methods, to provide a thorough understanding of configurations of the present disclosure. It will be apparent to those of ordinary skill in the art that specific details need not be employed, that example configurations may be embodied in many different forms, and that the specific details and the example configurations should not be construed to limit the scope of the disclosure.

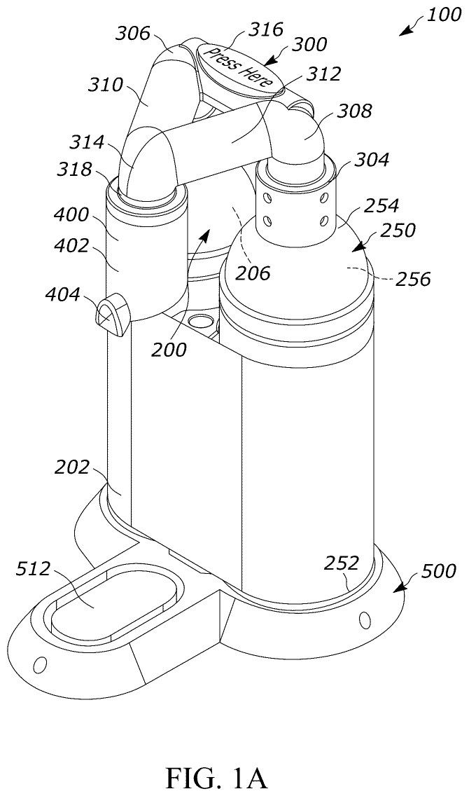

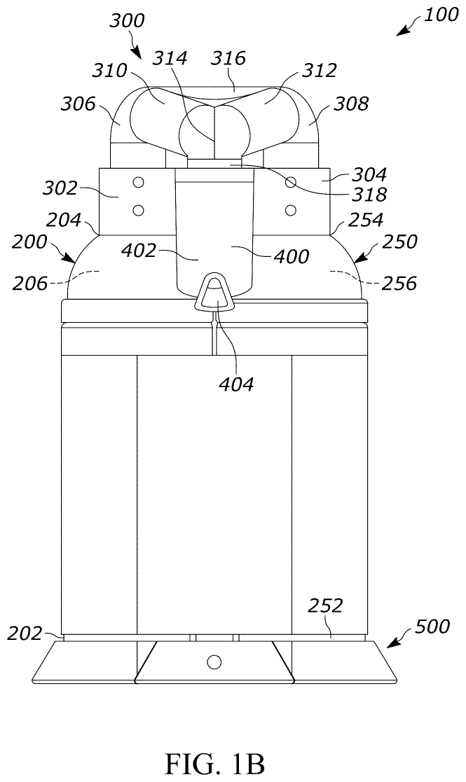

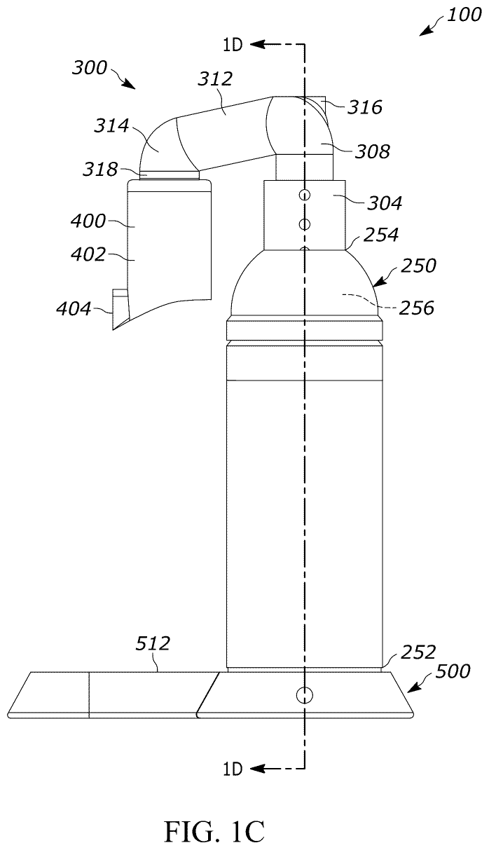

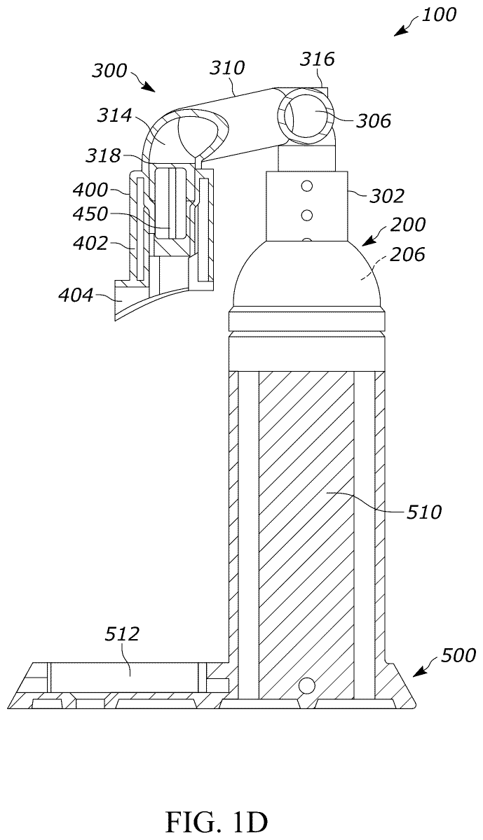

Referring to FIGS. 1A-ID, a Nitric Oxide (NO) formulation delivery device 100 is generally shown. The NO delivery device 100 may be used and configured to deliver NO as set forth herein. The NO delivery device 100 may include a first tank or cartridge 200, a second tank or cartridge 250, a plenum 300 in fluid communication with the cartridges 200, a nozzle 400 in fluid communication with the plenum 300, and a base 500 configured to receive the cartridges 200, 250.

With continued reference to FIGS. 1A-1D, the cartridges 200, 250 may each have a generally cylindrical shape defining a hollow interior. Each cartridge 200, 250 may extend from a bottom end 202, 252 to a top end 204, 254. The cartridges 200, 250 may each be generally the same size and shape. The cartridges 200, 250 may be formed of any suitable material, including, but not limited to a metal (e.g., aluminum, steel, etc.), a plastic, a rubber, a polymer, etc. The first cartridge 200 is configured to contain a first solution in its hollow interior, and the second cartridge 250 is configured to contain a second solution in its hollow interior. In some implementations, the first solution may comprise water, sodium nitrite, and a cationic surfactant. The second solution may comprise water, lactic acid and/or citric acid, and the cationic surfactant. One or both of the first and second solutions may additionally contain another agent, such as an antiseptic agent.

In one embodiment, a first solution may comprise approximately 100 mL of water, 10 g of sodium nitrite, and 1 g of cationic surfactant. A second solution may comprise approximately 100 ml of water, and an acidic solution of for example, 6 g of citric acid, and 3 g of cationic surfactant.

In one embodiment, a first solution may comprise approximately 100 ml of water, 10 g of sodium nitrite, 2 g of sodium bicarbonate, and 1 g of cationic surfactant. A second solution may comprise approximately 100 mL of water, 5 g of lactic acid, 8 g of citric acid, and 3 g of cationic surfactant.

In one embodiment, a first solution may comprise approximately 100 ml of water, 10 g of sodium nitrite, and 2 g of cationic surfactant. A second solution may comprise approximately 100 ml of water, 5 g of lactic acid, 6 g of citric acid, and 6 g of cationic surfactant.

In one embodiment, a first solution may comprise approximately 100 mL of water, 10 g of sodium nitrite, 2 g of sodium bicarbonate, and 1 g of cationic surfactant. A second solution may comprise approximately 100 ml of water, 5 g of lactic acid, 8 g of citric acid, and 6 g of cationic surfactant.

Each cartridge 200, 250 may include a foaming screen section 206, 256 near the top end 204, 254. The foaming screen sections 206, 256 may each include at least two of at least 200 mesh size screens perpendicular to the flow of the solutions, e.g., perpendicular to an axis extending from the bottom end 202, 252 to the top end 204 254. The screens are configured to provide sufficient flow shear orthogonal to the flow field to create a well-mixed and rich foam within the plenum 300 before the flow exits the nozzle 400. While screens are described, it should be understood that any suitable mechanism for providing sufficient flow shear may be implemented.

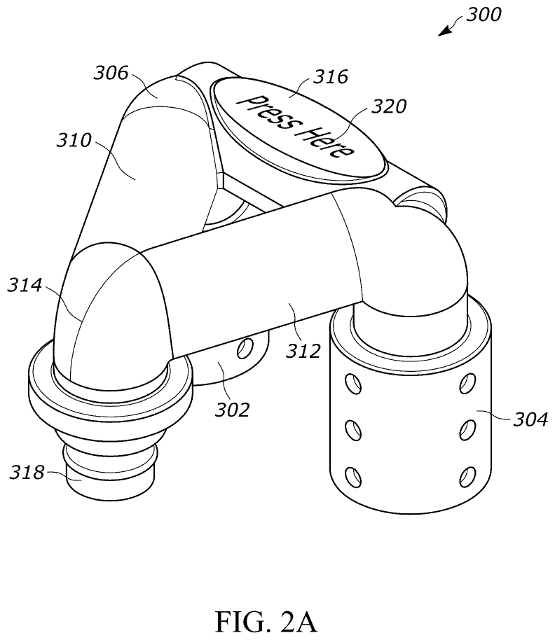

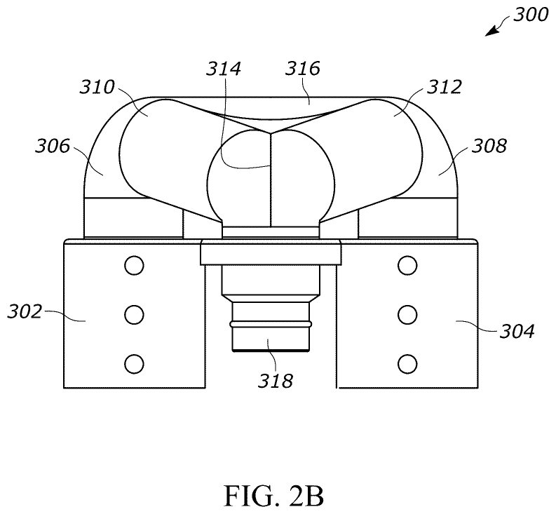

Referring to FIGS. 1A-2B, the plenum 300 includes a first connector 302 and a second connector 304. The first connector 302 is configured to engage with and connect to the first cartridge 200. The second connector 304 is configured to engage with and connect to the second cartridge 250. The connection between the connectors 302, 304 and the cartridges 200, 250 may be any suitable connection, such as a quick-connect mechanism, a screw-in mechanism, etc., and may facilitate fluid communication between the cartridges 200, 250 and the plenum 300.

The plenum 300 includes a first elbow portion 306 adjacent to and in fluid communication with the first connector 302 and a second elbow portion 308 adjacent to and in fluid communication with the second connector 304. The elbow portions 306, 308 curve from a generally vertical portion to a portion that is between 45 and 90 degrees relative to the vertical portion. The plenum 300 includes a first leg portion 310 adjacent to and in fluid communication with the first elbow portion 306 and a second leg portion 312 adjacent to and in fluid communication with the second elbow portion 308. In some implementations, the leg portions 310, 312 may extend in a direction that points slightly downward, as shown in FIGS. 1C and 2B. The length of each leg portion 310, 312 may be at least one flow diameter.

The plenum 300 includes a confluence portion 314 that is adjacent to and in fluid communication with each of the leg portions 310, 312. The confluence portion 314 receives an equal portion of the first solution and the second solution from the respective leg portions 310, 312. The confluence portion 314 is designed such that neither of the first solution nor the second solution can flow back into the leg portions 310, 312. The confluence portion 314 is located at least one flow diameter below the entrance to the leg portions 310, 312, i.e., in order to provide sufficient turbulent mixing where the leg portions 310, 312 and the elbow portions 306, 308 intersect.

The plenum 300 includes a head portion 316 extending between the elbow portions 306, 308. The head portion 316 is configured to facilitate movement of the plenum 300 in a downward direction in response to a force being applied to the head portion 316, e.g., by a user. The head portion 316 may include an indicator 320. In some implementations, the indicator 320 may be text, e.g., “Press Here.” In other implementations, the indicator 320 may be a logo, a higher-friction area, or any other suitable indicator.

The plenum 300 and/or cartridges 200, 250 may include any suitable means to pump the first and second solutions from their respective cartridges 200, 250 into the connectors 302, 304. Such means may include tubing, springs, chambers, and any other suitable components found in conventional pumping systems that may transform liquid into foam.

The plenum 300 includes a discharge portion 318 that is adjacent to and in fluid communication with the confluence portion 314. The leg portions 310, 312 and the discharge portion 318 may each include one or more flow tripper obstructions in their interior. The flow tripper obstructions are configured to create a turbulent flow. For example, the flow tripper obstructions may create a turbulent flow characterized by swirls and eddies generally associated with turbulent flow. The flow tripper obstructions may be studs, channels, ribs, screens, helix agitator, impeller, or any other suitable device. The flow tripper obstructions are configured to disrupt the flow of the first solution and the second solution to ensure complete, reactive mixing of the turbulent two solutions into a combined turbulent foam solution while traversing the discharge portion 318. The discharge portion 318 is configured to receive the nozzle 400 having a mixer 450.

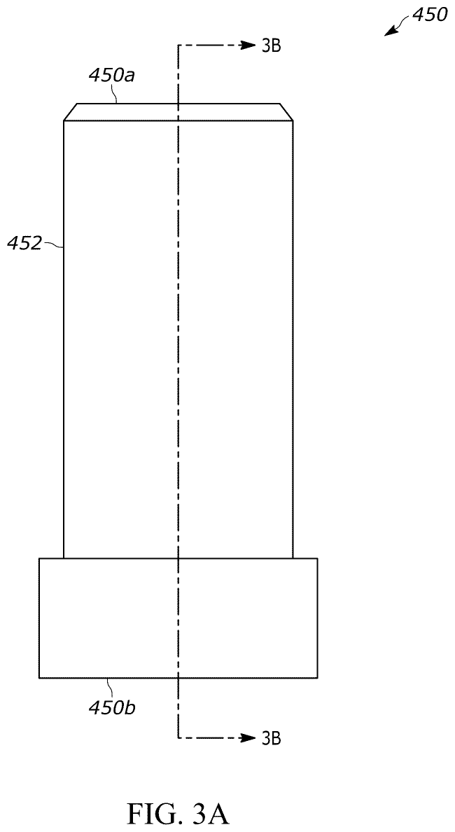

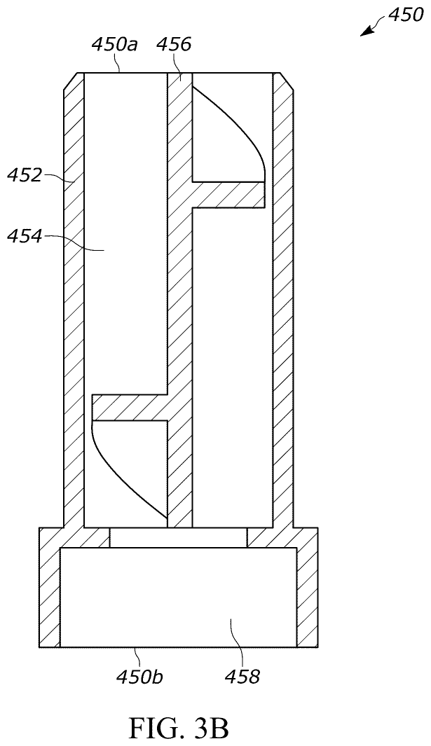

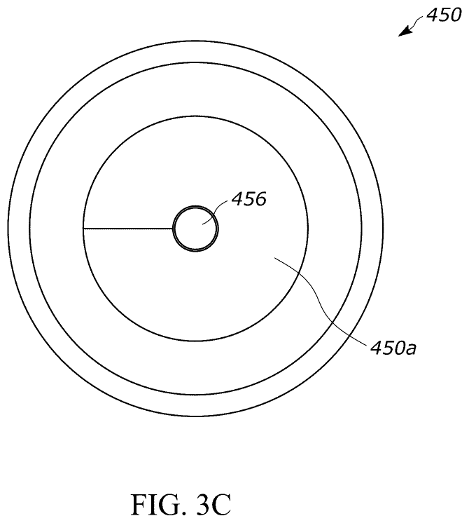

Referring to FIGS. 1A-1D and 3A-3C, the nozzle 400 is adjacent to and in fluid communication with the discharge portion 318 of the plenum 300. The nozzle 400 may be connected to the discharge portion 318 in any suitable manner, such as a quick-connect mechanism, a screw-in mechanism, etc. The nozzle 400 may include a body portion 402 configured to receive the combined turbulent foam solution and an exit port 404 configured to direct the combined turbulent foam solution in a desired direction of the combined turbulent foam solution along the body portion 402.

Referring to FIGS. 1D and 3A-3C, the nozzle 400 includes the mixer 450 in the body portion 402. In some implementations, the mixer 450 includes the flow tripper obstructions previously described, such as studs, channels, ribs, screens, helix agitator, impeller, etc. The mixer 450 extends from a proximal end 450a to a distal end 450b. The mixer 450 is configured to receive the combined turbulent foam solution from the confluence portion 314 at the proximal end 450a and is designed such that the combined turbulent foam solution cannot flow back into the confluence portion 314. In some implementations, the mixer 450 may be integrally formed with the plenum 300, e.g., within the discharge portion 318. The mixer 450 includes a portion 452 defining a hollow interior 454. The mixer 450 includes an agitator or impeller 456 disposed in the interior 454. The agitator 456 is configured to direct the combined turbulent foam solution from the proximal end 450a through at least one revolution throughout the interior 454 until passing through a flow-straightening portion 458 at or near the distal end 450b of the mixer 450. In some implementations, the combined turbulent foam solution flows through three revolutions along the agitator 456. In some implementations, the flow-straightening portion 458 may be a screen.

Referring to FIGS. 1A-ID and 4, the base 500 includes a first cartridge slot 502 and a second cartridge slot 504. The first cartridge slot 502 is configured to receive the first cartridge 200 and the second cartridge slot 502 is configured to receive the second cartridge 250. Each slot 502, 504 may include an engagement mechanism 506, 508 configured to secure the cartridges 200, 250 to the slots 502, 504. For example, the engagement mechanisms 506, 508 may each be a quick-connect mechanism, a screw-in mechanism, etc. The base 500 includes a support pillar 510 between the slots 502, 504 that extends vertically to provide support for the cartridges 200, 250. The support pillar 510 may include a first radial surface 510a and a second radial surface 510b. The radial surfaces 510a, 510b may be configured to correspond to the outer surface of each of the cartridges 200, 250.

The base 500 includes a catch basin 512 located below the exit port 404 of the nozzle 400. The catch basin 512 is configured to receive any residual combined turbulent foam solution that has exited the exit port 404. In some implementations, the catch basin 512 may include an absorbent pad configured to absorb any residual combined turbulent foam solution that has exited the exit port 404. The catch basin 512 allows the NO delivery device 100 to drain all or a majority of the prior dosage to ensure that the current dosage is accurate and consistent.

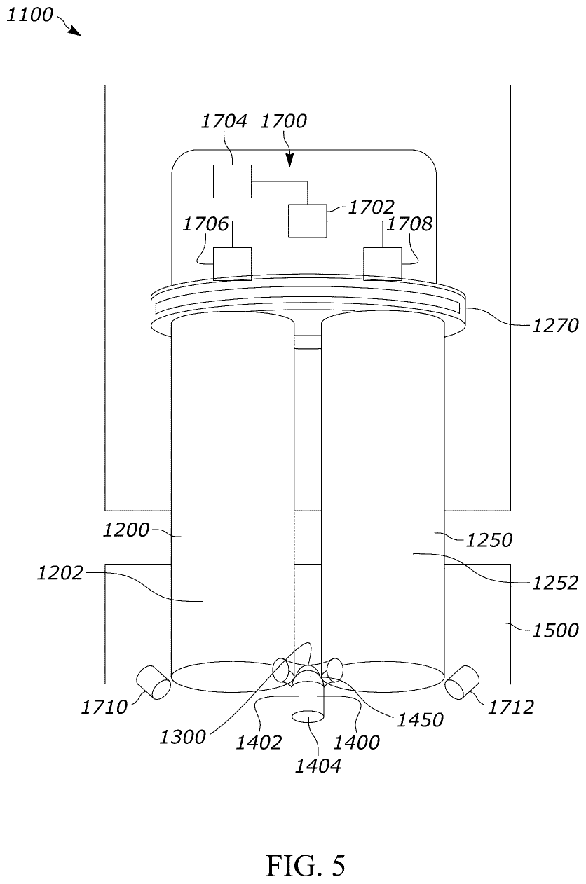

Referring to FIG. 5, a Nitric Oxide (NO) formulation delivery device 1100 is generally shown schematically. The NO delivery device 1100 may be substantially similar to the NO delivery device 100, but may enable the delivery of NO automatically (e.g., in response to a button or activation of a sensor) rather than via manual input. The NO delivery device 1100 may be used and configured to deliver NO as set forth herein. The NO delivery device 1100 may include a first tank or cartridge 1200, a second tank or cartridge 1250, a head 1270, a plenum 1300 in fluid communication with the cartridges 1200, 1250, a nozzle 1400 in fluid communication with the plenum 1300, and a base 1500 configured to receive the cartridges 1200, 1250.

The first cartridge 1200 is configured to contain a first solution in a hollow interior chamber 1202, and the second cartridge 1250 is configured to contain a second solution in a hollow interior chamber 1252. In some implementations, the first solution may comprise water, sodium nitrite, and a cationic surfactant. The second solution may comprise water, lactic acid and/or citric acid, and the cationic surfactant. The quantities and composition of the first and second solution may be the same as those previously described.

The NO delivery device 1100 includes an electronics system 1700 including a controller 1702 connected to a power source 1704 (e.g., battery). The controller 1702 may be any suitable controller, such as a processor, microprocessor, circuit board, application specific integrated circuit, etc. The controller 1702 may be connected to and configured to control a first cam pump 1706. The first cam pump 1706 is configured to exert a downward force on the head 1270. In some implementations, the controller may be connected to and configured to control a second cam pump 1708 to exert a downward force on the head 1270, or the first cam pump 1706 may drive a portion of the head 1270 down adjacent the first cartridge 1200 and the second cam pump 1708 may drive a portion of the head 1270 down adjacent the second cartridge 1250.

The electronics system 1700 includes a first sensor 1710 and, in some implementations, a second sensor 1712 directed at or near the nozzle 1400. The sensors 1710, 1712 may be light emitters and/or motion sensors configured to detect the presence of, e.g., a user's hands below the nozzle 1400. In some implementations, one or more of the sensors 1710, 1712 may be a button configured to receive an input from a user. When the sensors 1710, 1712 detect motion (e.g., a user's hands moving in front of the sensors or pressing a button), the sensors 1710, 1712 transmit a signal to the controller 1702 and the controller 1702 instructs the cam pump 1706 to operate to drive the head 1270 downward.

The plenum 1300 and/or the nozzle 1400 include a mixer 1450 including one or more flow tripper obstructions in their interiors. The mixer 1450 is configured to create a turbulent flow. For example, the mixer 1450 may create a turbulent flow characterized by swirls and eddies generally associated with turbulent flow. The mixer 1450 may include studs, channels, ribs, screens, helix agitator, impeller, or any other suitable device. The mixer 1450 is configured to disrupt the flow of the first solution and the second solution to ensure complete, reactive mixing of the turbulent two solutions into a combined turbulent foam solution while traversing the plenum 1300 and nozzle 1400. The plenum 1300 is configured to receive the nozzle 1400.

The plenum 1300 and/or cartridges 1200, 1250 may include any suitable means to pump the first and second solutions from their respective cartridges 1200, 1250 into the plenum 1300. Such means may include tubing, springs, chambers, and any other suitable components found in conventional pumping systems that may transform liquid into foam.

The nozzle 1400 is adjacent to and in fluid communication with the plenum 1300. The nozzle 1400 may be connected to the plenum 1300 in any suitable manner, such as a quick-connect mechanism, a screw-in mechanism, etc. The nozzle 1400 may include a body portion 1402 configured to receive the combined turbulent foam solution and an exit port 1404 configured to direct the combined turbulent foam solution in a desired direction of the combined turbulent foam solution along the body portion 1402. That is, the flow of the combined turbulent foam solution out of the exit port 1404 may be in a generally vertical direction, horizontal direction, or any other suitable direction. The nozzle 1400 may include any suitable mixer 1450, e.g., studs, channels, ribs, screens, agitators, impellers, flow-straightening portions, etc., as previously described.

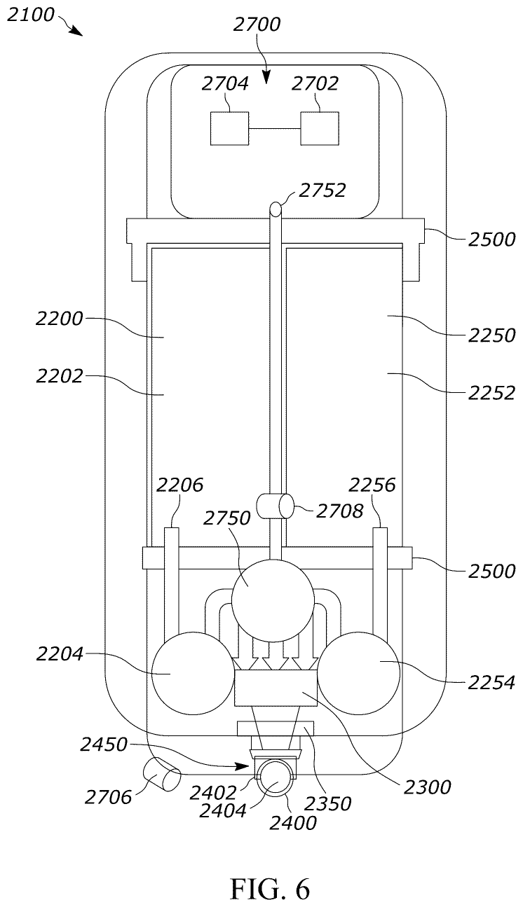

Referring to FIG. 6, a Nitric Oxide (NO) formulation delivery device 2100 is generally shown schematically. The NO delivery device 2100 may be substantially similar to the NO delivery device 100, but may enable the delivery of NO automatically (e.g., in response to a button or activation of a motion sensor) rather than via manual input. The NO delivery device 2100 may be used and configured to deliver NO as set forth herein. The NO delivery device 2100 may include a first tank or cartridge 2200, a second tank or cartridge 2250, a first mixing plenum 2300 in fluid communication with the cartridges 2200, 2250, a second mixing plenum 2350 in fluid communication with the first mixing plenum 2300, a nozzle 2400 in fluid communication with the second mixing plenum 2350, and a base 2500 configured to receive the cartridges 2200, 2250.

The first cartridge 2200 is configured to contain a first solution in a hollow interior chamber 2202, and the second cartridge 2250 is configured to contain a second solution in a hollow interior chamber 2252. In some implementations, the first solution may comprise water, sodium nitrite, and a cationic surfactant. The second solution may comprise water, lactic acid and/or citric acid, and the cationic surfactant. The quantities and composition of the first and second solution may be the same as those previously described.

The NO delivery device 2100 includes a first liquid pump 2204 (e.g., a peristaltic pump or any other suitable pump) in fluid communication with the first cartridge 2200 via a first pickup 2206 that extends into the first chamber 2202 and permits the first solution to flow to the first liquid pump 2204. The NO delivery device 2100 includes a second liquid pump 2254 (e.g., a peristaltic pump or any other suitable pump) in fluid communication with the second cartridge 2250 via a second pickup 2256 that extends into the second chamber 2252 and permits the second solution to flow to the second liquid pump 2254.

The NO delivery device 2100 includes a liquid air pump 2750 (e.g., a peristaltic pump or any other suitable pump) which obtains air through a pickup 2752. The liquid air pump 2750 receives the first solution from the first liquid pump 2204 and the second solution from the second liquid pump 2254 and pumps air, the first solution, and the second solution into the first mixing plenum 2300. In some implementations, the first liquid pump 2204, the second liquid pump 2254, and the liquid air pump 2750 may all be incorporated into a single pumping device.

In some implementations, the liquid air pump 2750 may inject air into the first solution and the second solution (e.g., into tubes connecting the liquid pumps 2204, 2254 to the first mixing plenum 2300) before the first solution, second solution, and air mix in the first mixing plenum 2300. In some implementations, the liquid air pump 2750 may inject air into the first mixing plenum 2300 simultaneously with the first solution and the second solution mixing with each other in the first mixing plenum 2300. In some implementations, the liquid air pump 2750 may inject air into the first mixing plenum 2300 after the first solution and the second solution have mixed with each other in the first mixing plenum 2300. Each of these implementations may be combined with one another, as suitable.

The NO delivery device 2100 includes an electronics system 2700 including a controller 2702 connected to a power source 2704 (e.g., battery). The controller 2702 may be any suitable controller, such as a processor, microprocessor, circuit board, application specific integrated circuit, etc. The controller 2702 may be connected to and configured to control the first liquid pump 2204, the second liquid pump 2254, and the liquid air pump 2750.

The electronics system 2700 includes a first sensor 2706 directed at or near the nozzle 2400. The sensor 2706 may be a light emitter and/or a motion sensor configured to detect the presence of, e.g., a user's hands below the nozzle 2400. In some implementations, the sensor 2706 may be a button configured to receive an input from a user. When the sensor 2706 detects motion (e.g., a user's hands moving in front of the sensor or pressing a button), the sensor 2706 transmits a signal to the controller 2702 and the controller 2702 instructs the first liquid pump 2204, the second liquid pump 2254, and the liquid air pump 2750 to operate.

The electronics system 2700 may include a second sensor 2708 configured to detect the levels of first solution and second solution in the first cartridge 2200 and second cartridge 2250, respectively. When the second sensor 2708 determines that one of the levels of first solution and second solution in the first cartridge 2200 and second cartridge 2250, respectively, fall below a predetermined threshold, the second sensor 2708 may send a signal to the controller 2702 indicating that the first solution and/or second solution are low and need to be refilled. In response, the controller 2702 may activate an LED or other indicator on the NO delivery device 2100.

The first mixing plenum 2300, the second mixing plenum 2350, and/or the nozzle 2400 include a mixer 2450 including one or more flow tripper obstructions in their interiors. The mixer 2450 is configured to create a turbulent flow. For example, the mixer 2450 may create a turbulent flow characterized by swirls and eddies generally associated with turbulent flow. The mixer 2450 may include studs, channels, ribs, screens, helix agitator, impeller, or any other suitable device. The mixer 2450 is configured to disrupt the flow of the first solution and the second solution to ensure complete, reactive mixing of the two solutions into a combined turbulent solution while traversing the plenums 2300, 2350 and nozzle 2400. The second mixing plenum 2350 is configured to receive the nozzle 2400.

The plenums 2300, 2350 and/or cartridges 2200, 2250 may include any suitable means to pump the first and second solutions from their respective cartridges 2200, 2250 into the plenums 2300, 2350. Such means may include tubing, springs, chambers, and any other suitable components found in conventional pumping systems that transform liquid into foam.

The nozzle 2400 is adjacent to and in fluid communication with the second mixing plenum 2350. The nozzle 2400 may be connected to the second mixing plenum 2350 in any suitable manner, such as a quick-connect mechanism, a screw-in mechanism, etc. The nozzle 2400 may include a body portion 2402 configured to receive the combined turbulent foam solution and an exit port 2404 configured to direct the combined turbulent foam solution in a desired direction of the combined turbulent foam solution along the body portion 2402. That is, the flow of the combined turbulent foam solution out of the exit port 2404 may be in a generally vertical direction, horizontal direction, or any other suitable direction. The nozzle 2400 may include any suitable mixer 2450, e.g., studs, channels, ribs, screens, agitators, impellers, flow-straightening portions, etc., as previously described.

While the NO delivery devices 100, 1100, 2100 are described separately, it should be understood that various aspects of each of these devices may be combined with one another, as suitable. As just one example, the automatic sensing and pump features of the NO delivery devices 1100, 2100 may be incorporated into the NO delivery device 100.

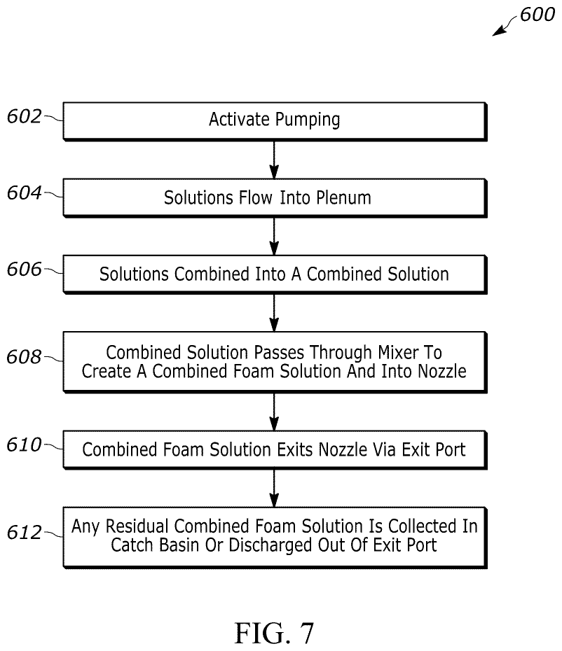

Referring to FIG. 7, a method 600 of operating the NO delivery device 100, 1100, 2100 is generally shown. In step 602, the pumping of the first and second solutions is activated. For the NO delivery device 100, this includes the plenum 300 moving in a downward direction in response to a force received (e.g., by a hand of a user) pressing down on the head portion 316. For the NO delivery devices 1100, this includes the sensors 1710, 1712 detecting the motion of a user's hands or detecting input from the user through a button. For the NO delivery devices 2100, this includes the sensors 2706 detecting the motion of a user's hands or detecting input from the user through a button. In step 604, the first solution and the second solution may flow from their respective cartridges 200, 250, 1200, 1250, 2200, 2250 and into the plenum 300, 1300, 2300. In step 606, the first solution and the second solution converge into a combined solution. In step 608, the combined solution passes through the mixer 450, 1450, 2450, creating the combined turbulent foam solution, and into the nozzle 400, 1400, 2400. In step 610, the combined turbulent foam solution exits the nozzle 400, 1400, 2400 via the exit port 404, 1404, 2404. In step 612, any residual combined turbulent foam solution is collected in the catch basin 512 of the base 500 or discharged out of the exit port 1404, 2404 as a trickle.

The NO delivery device 100, 1100, 2100 and the method 600 of operating the same allow a single pump (e.g., downstroke of the plenum 300 via a user pressing down on the head portion 316, activation of the cam pump 1706 in response to the sensors 1710, 1712 detecting motion of a user's hands, and/or activation of the first liquid pump 2204, the second liquid pump 2254, and the liquid air pump 2750 in response to the sensor 2706 detecting motion of a user's hands) to combine the first and second solutions into the combined turbulent foam solution. Each complete pump results in a consistent formulation delivery of the combined turbulent foam solution on the order of less than 5% variance of dosing between pumps. This consistency ensures that there is enough NO in each dosage resulting from a pump to be therapeutic. In some implementations, each pump produces between 0.6 g and 1.0 g of combined turbulent foam solution. In some implementations, each pump produces approximately 0.8 g of combined turbulent foam solution.

By containing the NO in a foam, the NO gas is suspended within the confines of each individual bubble of the foam. When the NO foam is applied topically to a skin wound, the bubbles rest on the skin and the wound and diffuse into the wound after the bubbles burst and the NO gas contained in these bubbles is released.

The terminology used herein is for the purpose of describing particular exemplary configurations only and is not intended to be limiting. As used herein, the singular articles “a,” “an,” and “the” may be intended to include the plural forms as well, unless the context clearly indicates otherwise. The terms “comprises,” “comprising,” “including,” and “having,” are inclusive and therefore specify the presence of features, steps, operations, elements, and/or components, but do not preclude the presence or addition of one or more other features, steps, operations, elements, components, and/or groups thereof. The method steps, processes, and operations described herein are not to be construed as necessarily requiring their performance in the particular order discussed or illustrated, unless specifically identified as an order of performance. Additional or alternative steps may be employed.

When an element or layer is referred to as being “on,” “engaged to,” “connected to,” “attached to,” or “coupled to” another element or layer, it may be directly on, engaged, connected, attached, or coupled to the other element or layer, or intervening elements or layers may be present. In contrast, when an element is referred to as being “directly on,” “directly engaged to,” “directly connected to,” “directly attached to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

The terms first, second, third, etc. may be used herein to describe various elements, components, regions, layers and/or sections. These elements, components, regions, layers and/or sections should not be limited by these terms. These terms may be only used to distinguish one element, component, region, layer or section from another region, layer or section. Terms such as “first,” “second,” and other numerical terms do not imply a sequence or order unless clearly indicated by the context. Thus, a first element, component, region, layer or section discussed above could be termed a second element, component, region, layer or section without departing from the teachings of the example configurations.

The foregoing description has been provided for purposes of illustration and description. It is not intended to be exhaustive or to limit the disclosure. Individual elements or features of a particular configuration are generally not limited to that particular configuration, but, where applicable, are interchangeable and can be used in a selected configuration, even if not specifically shown or described. The same may also be varied in many ways. Such variations are not to be regarded as a departure from the disclosure, and all such modifications are intended to be included within the scope of the disclosure.

What is claimed is:

1. A nitric oxide (NO) formulation delivery device comprising:
a first cartridge containing a first liquid solution and a first foaming device configured to transform the first liquid solution into a first foam solution;
a second cartridge containing a second liquid solution and a second foaming device configured to transform the second liquid solution into a second foam solution;
a plenum comprising:
a first leg portion in fluid communication with the first cartridge;
a second leg portion in fluid communication with the second cartridge; and
a confluence portion in fluid communication with the first leg portion and the second leg portion and configured to combine the first foam solution and the second foam solution to create a combined turbulent foam solution;
a nozzle in fluid communication with the confluence portion and configured to uniformly discharge the combined turbulent foam solution.

2. The NO formulation delivery device of claim 1, wherein the first leg portion is configured to receive the first foam solution, and the first leg portion includes one or more flow tripper obstructions configured to disrupt the flow of the first foam solution.

3. The NO formulation delivery device of claim 1, wherein the second leg portion is configured to receive the second foam solution, and the second leg portion includes one or more flow tripper obstructions configured to disrupt the flow of the second foam solution.

4. The NO formulation delivery device of claim 1, wherein the first solution comprises water, a nitrite, and a cationic surfactant.

5. The NO formulation delivery device of claim 1, wherein the second solution comprises at least one of water, a cationic surfactant, lactic acid, and citric acid.

6. The NO formulation delivery device of claim 1, wherein the plenum and nozzle are configured to discharge a consistent dose of the combined turbulent foam solution.

7. The NO formulation delivery device of claim 6, wherein the consistent dose of the combined turbulent foam solution is between approximately 0.6 g and 1.0 g.

8. The NO formulation delivery device of claim 1, further comprising a mixer disposed between the confluence portion of the plenum and the nozzle, the mixer comprising an agitator designed to direct the combined turbulent foam solution through at least one revolution as it flows through the mixer.

9. The NO formulation delivery device of claim 8, wherein the mixer includes a flow-straightening portion disposed between the agitator and the nozzle, the flow-straightening portion configured to straighten the flow of the combined turbulent foam solution.

10. The NO formulation delivery device of claim 8, wherein the mixer is designed to prevent the combined turbulent foam solution from flowing back into the plenum.

11. The NO formulation delivery device of claim 1, further comprising a base configured to receive the first cartridge and the second cartridge.

12. The NO formulation delivery device of claim 11, wherein the base includes a catch basin disposed below the nozzle, the catch basin configured to receive any residual combined foam or liquid solution discharged from the nozzle.

13. The NO formulation delivery device of claim 1, wherein the plenum includes a head portion connecting the first leg portion to the second leg portion.

14. A nitric oxide (NO) formulation delivery device comprising:
a first cartridge containing a first liquid solution and a first foaming device configured to transform the first liquid solution into a first foam solution;
a second cartridge containing a second liquid solution and a second foaming device configured to transform the second liquid solution into a second foam solution;
a plenum in fluid communication with the first cartridge and the second cartridge, the plenum being configured to combine the first foam solution and the second foam solution to create a combined turbulent foam solution;
one or more liquid pumps configured to operate automatically in response to a sensor to pump the first liquid solution and the second liquid solution from the first cartridge and the second cartridge to the plenum; and
a nozzle in fluid communication with the confluence portion and configured to discharge the combined turbulent foam solution.

15. The NO formulation delivery device of claim 14, wherein the first solution comprises water, a nitrite, and a cationic surfactant.

16. The NO formulation delivery device of claim 14, wherein the second solution comprises at least one of water, a cationic surfactant, lactic acid, and citric acid.

17. The NO formulation delivery device of claim 14, further comprising a mixer disposed between the plenum and the nozzle, the mixer comprising an agitator designed to direct the combined turbulent foam solution through at least one revolution as it flows through the mixer.

18. The NO formulation delivery device of claim 17, wherein the mixer is designed to prevent the combined turbulent foam solution from flowing back into the plenum.

19. A method comprising:
providing a nitric oxide (NO) formulation delivery device comprising:
a first cartridge containing a first liquid solution;
a second cartridge containing a second liquid solution;
a plenum in fluid communication with the first cartridge and the second cartridge, the plenum being configured to combine the first solution and the second solution to create a combined turbulent solution;
an air inlet configured to inject air into the first liquid solution and second liquid solution, or to inject air into the combined turbulent solution in order to produce a combined turbulent foam solution; and
a nozzle in fluid communication with the confluence portion and configured to discharge the combined turbulent foam solution;
simultaneously converting the first liquid solution from the first cartridge into the first foam solution, converting the second liquid solution from the second cartridge into the second foam solution, pumping the first foam solution and the second foam solution into the plenum, and mixing the first foam solution and the second foam solution in the plenum to create the combined turbulent foam solution; and
discharging the combined turbulent foam solution via the nozzle.

20. The method of claim 19, further comprising topically applying the combined turbulent foam solution to a skin wound.
